(19) United States
(12) Patent Application Publication
(10) Pub. No.: US 2014/0271600 A1
(43) Pub. Date: Sep. 18, 2014

(54) NON-CELLULAR BANDAGE, METHOD OF USING THE SAME, AND METHOD OF PREPARING THE SAME

(71) Applicants: Gabrielle Giet, Victoria (CA); Ian Milne, Victoria (CA); Dale Lane, Victoria (CA)

(72) Inventors: Gabrielle Giet, Victoria (CA); Ian Milne, Victoria (CA); Dale Lane, Victoria (CA)

(21) Appl. No.: 14/106,526

(22) Filed: Dec. 13, 2013

Related U.S. Application Data
(60) Provisional application No. 61/798,808, filed on Mar. 15, 2013.

(51) Int. Cl. A61L 15/40; A61L 15/38; A61L 15/22

(52) U.S. Cl. CPC A61L 15/40; A61L 15/225; A61L 15/38
USPC 424/94.5; 424/531

(57) ABSTRACT

There is provided a non-cellular bandage and a method of treating damaged tissue derived from a disease or symptom by applying the non-cellular bandage that includes serum, fibrin, and collagen. There is also provided a method of preparing a non-cellular bandage for treating damaged tissue derived from a disease or symptom including the steps of preparing serum, fibrin or an equivalent substitute, and collagen; mixing the serum, the fibrin, and the collagen to form a mixture; and forming a bandage by using the mixture. The non-cellular bandage facilitates the perfect environment in which to actively promote cellular regeneration and tissue repair, while simultaneously reducing immune rejections, scar formation and infection rates when it is applied to a biological tissue.

### BACKGROUND

The present invention is related to a non-cellular bandage, a method of using the same, and a method of preparing the same. More particularly, the present invention relates to a non-cellular bandage which comprises serum, fibrin and collagen. The non-cellular bandage facilitates the perfect environment in which to actively promote cellular regeneration and tissue repair, while simultaneously reducing immune rejections, scar formation and infection rates when it is applied to biological tissue.

Human tissue and the associated organ(s) can be damaged as a result of disease or wounds. Surgical or therapeutic treatment can be applied to the damaged tissue; however, such treatment may result in further damage if the treatment causes loss of tissue or biological function, infection or scarring. Furthermore, treatments that involve the use of materials (biological or otherwise) that are not fully compatible with the damaged tissue may lead to a rejection response or an adverse unnecessary immune response.

For example, in the area of ophthalmology, the cornea is subject to corneal disease as a result of infection or injury. This, in turn, may lead to a loss of vision. Currently, over 10 million individuals worldwide are suffering from a loss of vision due to corneal disease and/or trauma. In the US, an estimated 42,000 corneal grafts are performed annually as a treatment for corneal disease. This treatment often involves using optical tissue from cadavers or living donors; however, graft rejection can occur, and failure rates are significant. To limit rejection and failure rates, steroids are used with donor-sourced corneal material, but rejection can still occur and failure rates, though improved, are still significant. Tissue engineered materials and fully synthetic prostheses (without biological activity) have been developed, but their use has been limited to cases where human donor tissue fails repeatedly or cannot be used. To date, and prior to this invention, no biological technology has existed for the auto regeneration of human tissue.

Presently, ocular surface treatments and research for corneal disease have focused on the following therapies:

(1) Keratoplasty (cornea transplant). This method is limited because patients with dry eye or pro-inflammatory ocular conditions are not good candidates for keratoplasty. In addition, the trauma caused to the surface of the eye during surgery can pose significant risk to the patient's natural eye. Additional risks include rejection, infection, scarring and/or the development of opacities from surgical trauma.

(2) Amniotic membrane graft sutured to the ocular surface or held to the surface using a plastic ring. This method also requires surgery. Thus, it poses risks similar to keratoplasty.

(3) Surgical reconstruction of the eyelids. This method also poses a high risk and often negatively affects the patient's visual acuity as well as the overall health of the ocular surface.

(4) Human Serum Eyedrops both autologous and homologous. The serum eyedrops flow off the eye and cannot be sustained for a long period of time. If a patient requires therapeutic eyedrops then there must be an interval between application of the serum eyedrops and the therapeutic agent. Also there is a possibility of patient error that leads to side effects due to an overdose of the therapeutic agent.

(5) Human Amniotic Fluid eyedrops. The amniotic eyedrops also flow off the eye. If a patient requires therapeutic eyedrops then, as explained above, there must be an interval between application of the serum eyedrops and the therapeutic agent. Also there is a possibility of patient error that leads to side effects due to an overdose of the therapeutic agent.

(6) Frequent use of synthetic tear replacement eyedrops and lubricating ointments. This method may be dependant not just on compliance but also on the patient's own technique. The problem with these products is that they only treat symptoms; they do not cure the disease. If a patient requires therapeutic eyedrops, then, as explained above, there must be an interval between application of the synthetic tear replacement eyedrops and the therapeutic agent. Also there is a high possibility of patient error that leads to side effects due to an overdose of the therapeutic agent. Furthermore, eyedrops and ointments flow off the eye.

(7) Use of steroid and immunosuppressant medications applied topically or injected. These methods may be toxic to both the eye and body and, therefore, present a significant risk of adverse reaction.

(8) Collagen Shields. This method requires the insertion of a foreign body into the eye posing a substantial risk of infection. The collagen is not vital so the therapeutic effects are not guaranteed.

(9) Bandage Contact lens. This method also requires the insertion of a foreign body into the eye posing a substantial risk to an already fragile ocular condition. In addition, the bandage contact lens can act as a barrier to therapeutic agents.

(10) Keratoprosthetic Devices. This method is related to the use of specific keratoprosthetic devices, such as Boston K-Pro Device (a.k.a. Dohlman-Doane Device), Boston Prose Device (a.k.a. prosthetic replacement of the ocular surface ecosystem) and/or Osteo-Odontic Keratoprosthesis. However, current keratoprosthetic technologies still require the insertion of a foreign, synthetic object into the eye providing the opportunity for bacterial infection and inflammation. These devices are dependent on patient compliance for successful implementation, but they are not compatible with ongoing tissue changes in the eye. The Boston K-Pro Device may involve extensive surgical intervention, not the least of which is pronounced suturing of the device onto the ocular surface. The Osteo-Odonto Keratoprosthesis is a high risk surgical procedure that is severely destructive to the natural eye and requires destructive harvesting of the patient's (or donor's) own tooth.

(11) Research. Current research focuses on the use of tissue engineered materials including variants on the theme of stem cell replacement and collagen based corneal prosthesis, most of which are dependent on surgical grafting.

In view of the above, a new therapeutic approach(es) for protecting or otherwise treating damaged tissue (e.g., corneal tissue), or tissue susceptible to degeneration, damage or infection, is needed. In particular, a new approach that requires no incision or immunological suppression; helps decrease, rather than increase the likelihood of infection; provides nutrients and vitality to help restore tissue; and involves material that is not perceived by the body as a foreign object is needed.

One of the inventors of this invention suffered from Toxic Epidermal Necrolysis, which is a severe form of Stevens Johnson Syndrome. It involves permanent damage to the mucus membranes resulting in keratoconjunctivitis sicca (severe dry eye) and the destruction of the lacrimal glands, meibomian glands and punctae. This, in turn, often causes chronic and severe ocular surface disease, which typically leads to a loss of vision due to limbal stem cell deficiencies, corneal opacities, scar tissue, neovascularization of the cornea, conjunctival symblepharon and/or ptergyium. This condition can also result in the destruction of the eyelids leading to a host of scar related conditions of the inner eyelids (fornix, tarsus, conjunctiva) including but not limited to keratinization, entropian, ectropian, distichiasis and lagothalmos. Over the course of 11 years, the treatments have had little to no effect on the progression of the disease or the symptoms related thereto.

### SUMMARY OF THE INVENTION

In general, the present invention involves a new combination of materials that can be used for treating diseases or symptoms relating to said diseases without the problems associated with the aforementioned, current therapeutic options. The present invention is not limited to treatment of external tissue.

In accordance with one aspect of the present invention, the benefits and advantages associated with the present invention are achieved by a non-cellular bandage comprising serum, fibrin, and collagen. The serum may be human serum or veterinary serum. The human serum may comprise human autologous serum and/or human homologous serum. The serum and the fibrin may be derived from the same plasma. The collagen may be type I collagen or type III collagen. The bandage may further comprise a therapeutic agent and/or factor XIII. The bandage may take the form of a sheet, graft, patch, lens, plug, wrap, dressing, strip, gel, ointment, drops or spray.

In accordance with another aspect of the present invention, the benefits and advantages associated with the present invention are achieved by a method of treating damaged tissue that involves applying a non-cellular bandage comprising serum, fibrin, and collagen. The damaged tissue is derived from a disease or symptom that may be the result of a bacterial, viral, autoimmune or genetic condition, trauma; thermal or chemical burns; ocular surface disease including cornea transplant, keratitis, ulcer, dry eye and lagothalmos sequelae, aniridia, keratoconus or pterygium, limbal stem cell deficiency, corneal opacity, corneal dystrophies, meibomian gland dysfunction, keratinization of the inner lids or trichiasis; infection or trauma (lacerations); sequelae from surgical trauma; reconstructive or cosmetic surgery; necrotising fasciitis; internal surgical site in need to packing or wrapping; external surgical site in need to packing or wrapping; and breached skin. The serum may be human serum or veterinary serum, and the human serum may comprise human autologous serum and/or human homologous serum. The serum and the fibrin may be derived from the same plasma. The collagen may be type I collagen or type III collagen. The bandage may further include a therapeutic agent and/or factor XIII. The bandage may take the form of a sheet, graft, patch, lens, plug, wrap, dressing, strip, gel, ointment, drops or spray.

In accordance with still another aspect of the present invention, the benefits and advantages associated with the present invention are achieved by a method of preparing a non-cellular bandage for treating damaged tissue. The method involves: preparing serum, fibrin, and collagen; mixing the serum, the fibrin, and the collagen; and forming a bandage with the mixture. The serum may be human serum or veterinary serum, and the human serum may comprise human autologous serum and/or human homologous serum. The serum and the fibrin may be derived from the same plasma. The collagen may be type I collagen or type III collagen. The method may further include adding a therapeutic agent and/or factor XIII into the mixture. The bandage may take the form of a sheet, graft, patch, lens, plug, wrap, dressing, strip, gel, ointment, drops or spray.

These and other aspects of the present invention will be appreciated by one of ordinary skill in the art upon reading and understanding the following specification.

The non-cellular bandage according to the present disclosure has several advantages. For example, this dissolvable and self-adhesive bandage will provide a sterile, gas permeable, nutrient rich and vital environment that will promote, maximize and increase the rate of cellular regeneration in order to prevent infection and achieve rapid healing. This in turn will lead to higher surgical success rates, decreased (faster) recovery times: surgical and therapeutic, decreased infection rates, and elimination of scar tissue formation (softening and reduction of existing scar tissue). In addition the combined effects of the use of this non-cellular bandage material will lead to substantial decreases in health care costs.

### BRIEF DESCRIPTION OF THE DRAWINGS

FIG. 1 is a photo of a blood sample collected in a non-additive vacutainer tube.

FIG. 2 is a photo of the vacutainer tubes containing the blood.

FIG. 3 is a photo of serum collected from the centrifuged vacutainer tubes under 3500 rpms for 15 minutes.

FIG. 4 is a photo of serum of FIG. 3 further including collagen and fibrinogen.

FIG. 5 is a photo of a solution of serum, collagen and fibrinogen in a dish before thrombin is added to form fibrin.

FIG. 6 is a photo of serum, collagen and fibrinogen after thrombin is added and stabilized by factor XIII.

FIG. 7 is a photo of a bandage according to exemplary embodiments of the present invention.

FIG. 8 is a photo of the bandage of FIG. 7 placed on a patient's hand.

FIG. 9 is a photo of a frozen bandage according to exemplary embodiments of the present invention.

### DETAILED DESCRIPTION OF THE DISCLOSURE

As described above, the present invention is directed to a non-cellular bandage comprising serum, fibrin or an equivalent substitute, and collagen, as well as a method of using and making same. The bandage may further comprise a therapeutic agent.

Serum includes diverse components, for example, non-coagulating proteins, electrolytes and immunoglobulins. Therefore, it is a superior choice for the non-cellular bandage according to exemplary embodiments of the present invention. More specifically, serum contains many of the nutrients and vitality required for rapid cellular regeneration. The immunoglobulins included in a serum help to decrease the likelihood of disease based or post operative infection. Serum can be obtained from whole blood and the method of separating serum from a whole blood is not limited. For example, serum can be obtained by allowing whole blood to clot in a glass vessel (contact activation) and then removed from the clot by centrifugation. During the clot formation from whole blood, many of the clotting proteins are totally consumed and the remainders are reduced to variable extents, and in some cases inactivated. When the non-cellular bandage is used for a human patient, it is desirable to use human serum. Likewise, it is preferable to use veterinary serum if the non-cellular bandage is used for an animal. Additionally, when using the non-cellular bandage for an animal, it is most preferable to use serum from the same species of animal.

The human non-cellular bandage may include human autologous serum and/or human homologous serum. Autologous serum is derived from the same patient on whom the bandage is to be used. Homologous serum is derived from a human donor preferably having the same blood type and Rh factor as the patient. However, a compatible Rh factor may be of less concern.

In one alternative embodiment of the present invention, plasma may be used as the source of a serum and fibrin. Plasma includes both serum and fibrinogen which can form fibrin by introducing thrombin. Plasma may be obtained by adding an anticoagulant to whole blood and separating cells from the whole blood. The anticoagulant chelates specific ions, thereby inhibiting the clotting process and leaving the clotting mechanisms intact. However, the addition of the anticoagulant may reduce the vitality of the plasma compared to that of the serum. Accordingly, the exemplary embodiment that uses serum and fibrin (instead of plasma) may be preferable given that the serum is free of chemicals thereby avoiding the possibility of adverse reactions. The use of serum will actively promote cellular regeneration, tissue repair and eliminate the formation of scar tissue in addition to prevent cellular deterioration and minimize scar formation. The use of serum, compared to plasma, will also minimize microclot formation.

Collagen is one of the body's key natural resources and a component of biological tissue that benefits all stages of the wound healing process. Collagen is also a structural protein and, in accordance with the preferred embodiments of the present invention, an additional constituent of the bandage. For the purpose of the present invention, collagen can be obtained from several sources such as animal collagen, plant or marine collagen, and human collagen. However, it is preferable to use human collagen, if the patient is human, in order to maximize the healing effects of the bandage. Also, there are several types of collagen, such as Type I through Type XXVIII collagen. Each of these collagen types is a potential collagen source for the non-cellular bandage in accordance with exemplary embodiments of the present invention. However, one skilled in the art will appreciate that Type I and Type III collagen are the most common and abundant types of collagen found in the human body.

Fibrin is a fibrous, non-globular protein involved in the clotting of blood. It is understood that fibrin may be formed by cleaving fibrinogen using the protease thrombin, whereby a fibrin polymer mesh is produced. In accordance with the preferred embodiment of the present invention, fibrin is an additional constituent of the non-cellular bandage. When preparing the non-cellular bandage for a human patient, it is preferable that the fibrin is human fibrin. When preparing the non-cellular bandage for another species, it is preferable to use fibrin from the same species.

One of ordinary skill in the art will appreciate the cross linking aspect of the combination of collagen and fibrin in that the collagen threads woven through the fibrin network will work to hold the serum within the structure of the non-cellular bandage. In order to stabilize fibrin network and form a cross-linking structure in the non-cellular bandage, factor XIII can be added in the bandage.

In accordance with another exemplary embodiment of the present invention, the non-cellular bandage further includes a therapeutic agent. The therapeutic agent may involve, but is not limited to, medications and/or vitamins. Moreover, the therapeutic agent may be in powder or crystallized form. The bandage can be impregnated with therapeutic agent after the constituents of the bandage are combined. In a preferred embodiment of the present invention, the therapeutic agent can be impregnated into the bandage after the serum, fibrin and collagen are combined.

The bandage of the present invention is non-cellular. As such, no cellular byproducts form during the storage of the bandage.

The non-cellular bandage according to the present invention may take the form of a sheet, graft, patch, lens, plug, wrap or dressing. For example, the bandage can be formed into sheets, and primarily used for repairing large tissue. The bandage may also be in the form of an ocular dressing or lens design for repairing of the eye tissue (including inner eyelid tissue). The bandage may be in the form of a plug for repairing organs that include a cavity, such as the ear and certain glands. In addition, the bandage will provide the option for variable viscosities, thereby allowing for additional formulations and applications as a gel, ointment, spray or eye drop.

The non-cellular bandage according to exemplary embodiments of the present invention is self adhesive, dissolvable and non-destructive of the tissue on which it is placed, and thus there may be no threat of rejection and no immunosuppressant medications may be required. The bandage also provides a gas permeable environment, while simultaneously reducing oxidation. Where oxidation of the tissue substantially reduced, rapid healing is encouraged with fewer post operative or therapeutic complications.

In an application to an ocular surface, for example, the non-cellular bandage according to exemplary embodiments of the present invention allows for a non-surgical method and will act as both a bandage and graft effective in promoting healing of persistent corneal epithelial defects with ulceration, management of inflammation, reduction in angiogenises and scarring. The non-cellular bandage can be used for the restoration of the epithelial phenotype, all forms of ocular surface reconstruction and repair including but not limited to: sequelae to keratoconjunctivitis sicca, corneal opacities, corneal ulcers and performations, dellen formation, chemical and thermal burns, removal of pterygia, symblepharon or granuloma, corneal calcifications, ocular cicatrical pemphigoid, aniridia, atopic keratitis, idiopathic stem cell deficiency and fornix reconstruction.

As mentioned, the present invention is applicable to the treatment of ocular conditions listed above and symptoms associated therewith. However, it will be apparent to those skilled in the art that the present invention is applicable to the treatment of other disorders or symptoms, as outlined below.

Ophthalmology: The bandage may be designed to have a specific shape in the form of a wrap, such as a “petal or butterfly,” with individual overlapping segments (like leaves or petals) that can be wrapped around the eye. The use of the non-cellular bandage may be useful for treating damaged tissue derived from thermal or chemical burns; cornea transplant or repairs; keratitis; ulcer, infection or trauma (lacerations); sequelae from surgical trauma; dry eye and lagothalmos sequelae; aniridia, keratoconus or pterygium; regeneration of the limbus; corneal opacity; corneal dystrophies; scar tissue; meibomian gland dysfunction; keratinization of the inner lids; trichiasis; or reconstructive or cosmetic surgery.

Dermatology: In the area of dermatology, the use of the non-cellular bandage may also be useful for treating damaged tissue derived from burns by preventing infection, skin regeneration and/or minimizing scar formation; necrotising fasciitis; removal of cancerous tissue; breached skin, e.g. on top of stitches; reconstructive or cosmetic surgery: wounds such as deep cuts or gashes.

Pulmonology/Respiratory: External dressings on lungs or trachea.

Cardiology: External wrapping for the heart or blood vessels.

Orthopedics: Packing a surgical site after joint replacement or removal of tumors or any like surgical procedure.

Neurology: Packing and wrapping a surgical site after the removal of tumors or any like surgical procedure. When the bandage is applied to brain, it can be disposed between brain and skull during the surgical procedures, just before skull is reattached.

Urology: Packing and wrapping a surgical site after the removal of tumors or any like surgical procedure including testicular and prostate cancers.

Gastroenterology: (1) Bandage material can be inserted into the colon or intestines before a colonoscopy/endoscopy to prevent damage from scope. Pack with material after the procedure; (2) Packing and wrapping a surgical site after the removal of tumors or any like other surgical procedure. When applied to an intestine, it is preferable to apply the bandage first, before the colonoscopy/endoscopy, to prevent damage caused by the endoscope. The bandage can be also used for packing areas after removing the intestine, gallbladder, appendix, bladder, esophagus, or other organs.

Gynacology: Packing and wrapping a surgical site after the removal of tumors or any like surgical procedure; wrap the uterus after removal of endometriosis, polyps or fibroids.

Hepatology: Packing and wrapping a surgical site after the removal of tumors or any like surgical procedure.

Organ Transplantation

Furthermore, the present invention is also applicable for organ transportation. When transporting an organ for transplantation, it is preferable to wrap the organ with a bandage that includes autologous serum from the donor. When the bandage is used after an organ transplant, it is preferable to pack surgical area with a bandage that includes autologous serum from the recipient.

Other Applications

The bandage can be used for packing the internal cavities in an organ, such as chest/thorax. The bandage can be used for treating wounds by packing the wounds with the bandage. The bandage can be used for curing deafness related to ear drum or tissue damage by using the bandage in the form of a plug and placed into the ear. The bandage can also be used in the form of a plug and placed in the nose during surgeries. The bandage can be applied to spinal cord injuries by wrapping the surgical area as part of serum fluid treatment (wrap and gently flow of electrically stimulated serum). The bandage can be applied to an amputation site by wrapping the affected area. When the bandage is used in neonatal or pediatric situations, it may eliminate damage to premature skin when removing the bandage.

The constituents of the bandage of the present invention can be made, for example, by mixing serum, collagen and fibrin (or equivalent substitute) in any of the following preferred examples:
Autologous Serum+Collagen+Fibrin
Autologous Serum+Homologous Serum+Collagen+Fibrin
Autologous Serum+Therapeutic Agent+Collagen+Fibrin
Autologous Serum+Homologous Serum+Therapeutic Agent+Collagen+Fibrin

In each of the above examples, factor XIII can be also added. The bandage of the present invention can take the form of a sheet. It can be cut to predetermined sizes and thicknesses depending on the size of tissue. For internal and external use, the material for the bandage may be used and packaged without an attached bandage or dressing. For external uses the bandage may also be packaged with a sterile bandage dressing larger than the size of the bandage material, to allow for an adhesive rim if necessary.

It is preferable to prepare the non-cellular bandage, according to one embodiment of the present invention, within a closed system. It is preferable that the total processing time from drawing blood to freezing and storing the bandage material does not exceed 3 hours in order to maintain the vitality of the serum.

It is also preferable to store the bandage between temperatures of −15° C. and −28° C. It is not desired to store the bandage above −15° C. or below −30° C. Once thawed, a medical practitioner can place the bandage directly on the area to be treated. In a surgical situation, the medical practitioner may then cut through the material as part of the surgical incision and slide the bandage inside the body. As discussed above, the bandage material may be placed on the organ or area being repaired or excised, with the procedure taking place as normal, but through the bandage material.

### Example

A non-cellular bandage according to the present invention was made by the following process. A whole blood sample was collected from a patient as shown in FIG. 1 by using non-additive vacutainer tubes. The collected blood sample was centrifuged at 3500 rpm for 15 minutes to obtain serum (FIG. 3). 8 ml of serum was then mixed with 0.1 gms of collagen and 0.025 gms of fibrinogen (FIG. 4). The mixture of serum, collagen and fibrinogen was warmed to 37 degrees Celsius. The mixture was then poured into a small dish (FIG. 5). 10 IU of thrombin was added to the mixture to form fibrin. The collagen and fibrin mesh is further stabilized by factor XIII (FIG. 6). As a result, a bandage according to an exemplary embodiment of the present invention was obtained (FIG. 7).

In order to check the self-adhesiveness of the bandage, it was disposed on the top of a hand and stayed in place as the hand was moved (FIG. 7). The bandage according to the present disclosure shows a good adhesiveness to a skin.

It is to be understood that the above-described bandage and methods are merely illustrative embodiments of the principles of this disclosure, and that other compositions and methods for using them may be devised by one of ordinary skill in the art, without departing from the spirit and scope of the disclosure. It is also to be understood that the disclosure is directed to embodiments both comprising and consisting of the disclosed parts.

What is claimed is:

1. A non-cellular bandage comprising:
serum,
fibrin, and
collagen.

2. The bandage of claim 1, wherein the serum is human serum or veterinary serum.

3. The bandage of claim 2, wherein the human serum comprises human autologous serum.

4. The bandage of claim 2, wherein the human serum comprises human homologous serum.

5. The bandage of claim 2, wherein the human serum comprises human autologous serum and human homologous serum.

6. The bandage of claim 1, wherein the serum and the fibrin are derived from the same plasma.

7. The bandage of claim 1, wherein the collagen is type I collagen or type III collagen.

8. The bandage of claim 1, wherein the bandage further comprises a therapeutic agent.

9. The bandage of claim 1, wherein the bandage further comprises factor XIII.

10. The bandage of claim 1, wherein the bandage takes the form of a sheet.

11. The bandage of claim 1, wherein the bandage takes the form of a graft.

12. The bandage of claim 1, wherein the bandage takes the form of a patch.

13. The bandage of claim 1, wherein the bandage takes the form of a lens.

14. The bandage of claim 1, wherein the bandage takes the form of a plug.

15. The bandage of claim 1, wherein the bandage takes the form of a wrap.

16. The bandage of claim 1, wherein the bandage takes the form of a dressing.

17. The bandage of claim 1, wherein the bandage takes the form of a strip.

18. The bandage of claim 1, wherein the bandage takes the form of a gel.

19. The bandage of claim 1, wherein the bandage takes the form of an ointment.

20. The bandage of claim 1, wherein the bandage takes the form of drops.

21. The bandage of claim 1, wherein the bandage takes the form of a spray.

22. A method of treating damaged tissue, comprising:
applying a non-cellular bandage to damaged tissue,
wherein the non-cellular bandage comprises:
serum,
fibrin, and
collagen.

23. The method of claim 22, wherein the damaged tissue is the result of disease or symptom selected from the group consisting of a bacterial, viral, autoimmune or genetic condition, trauma; thermal or chemical burns; ocular surface disease including cornea transplant, keratitis, ulcer, dry eye and lagothalmos sequelae, aniridia, keratoconus or pterygium, limbal stem cell deficiency, corneal opacity, corneal dystrophies, meibomian gland dysfunction, keratinization of the inner lids or trichiasis; infection or trauma (lacerations); sequelae from surgical trauma; reconstructive or cosmetic surgery; necrotising fasciitis; internal surgical site in need to packing or wrapping; external surgical site in need to packing or wrapping; and breached skin.

24. The method of claim 22, wherein the serum is human serum or veterinary serum.

25. The method of claim 24, wherein the human serum comprises human autologous serum.

26. The method of claim 24, wherein the human serum comprises human homologous serum.

27. The method of claim 24, wherein the human serum comprises human autologous serum and human homologous serum.

28. The method of claim 22, wherein the serum and the fibrin are derived from the same plasma.

29. The method of claim 22, wherein the collagen is type I collagen or type III collagen.

30. The method of claim 22, wherein the bandage further includes a therapeutic agent.

31. The method of claim 22, wherein the bandage further includes factor XIII.

32. The method of claim 22, wherein the bandage takes the form of sheet, graft, patch, lens, plug, wrap, dressing, strip, gel, ointment, drops or spray.

33. A method of preparing a non-cellular bandage, comprising:
preparing serum, fibrin, and collagen,
mixing the serum, the fibrin, and the collagen, and
forming a bandage by using the mixture.

34. The method of claim 33, wherein the serum is human serum or veterinary serum.

35. The method of claim 34, wherein the human serum is human autologous serum.

36. The method of claim 34, wherein the human serum is human homologous serum.

37. The method of claim 34, wherein the human serum is human autologous serum and human homologous serum.

38. The method of claim 33, wherein the serum and the fibrin are derived from the same plasma.

39. The method of claim 33, wherein the collagen is type I collagen or type III collagen.

40. The method of claim 33, wherein the method further comprises adding a therapeutic agent to the mixture.

41. The method of claim 33, wherein the method further comprises adding factor XIII.

42. The method of claim 33, wherein the bandage takes the form of sheet, graft, patch, lens, plug, wrap, dressing, strip, gel, ointment, drops or spray.
